(19) United States
(12) Patent Application Publication
(10) Pub. No.: US 2005/0175681 A1
(43) Pub. Date: Aug. 11, 2005

(54) Liposomal vitamin a and method of preparation

(76) Inventors: Jianming Chen, Shanghai (CN); Shen Gao, Shanghai (CN); Shaomin Wei, Shanghai (CN); Hulfen Lin, Shanghai (CN); Yangmei Zhang, Shanghai (CN); Fel Guan, Shanghai (CN); Yanqiang Zhong, Shanghai (CN); Huiliang Li, Shanghai (CN); Qing Shi, Shanghai (CN); Yiguang Guo, Shanghai (CN); Luo Lu, Shanghai (CN)

Correspondence Address: THE LAW OFFICE OF RANDALL T. ERICKSON, P.C., 425 WEST WESLEY STREET, SUITE 1, WHEATON, IL, 60187, US

(21) Appl. No.: 10/517,088

(22) PCT Filed: Jan. 3, 2003
(86) PCT No.: PCT/CN03/00005

(30) Foreign Application Priority Data
Jun. 6, 2002 (CN) 02111967.8

(51) Int. Cl. A61K 9/127; A61K 31/07

(52) U.S. Cl. 424/450; 514/725

(57) ABSTRACT

The invention relates to liposomal vitamin A and method of its preparing. The liposome containing vitamin A as active agent, carriers (supporter) and lipids as adjuvents and liposome-forming materials. The process comprises: adding vitamin A and lipids to carriers, forming a liposomal vitamin A in the form of solid, then rehydrating to give a liposomal dispersion. The process can improve the stability of vitamin A and liposomal vitamin A. The present liposome is useful for the manufacture of pharmaceutical and cosmetic formulation.

### TECHNICAL FIELD

This invention relates to the field of pharmaceutical and cosmetics production, mainly referring to a kind of Liposomes which contains Vitamin A and the method of its preparation.

### BACKGROUND OF THE INVENTION

Vitamin A is one of the essential nutriments of human body, which likes a hormone for the regulation of cell differentiation, growth and development and for the maintenance of metabolic balance and internal environment homeostasis, as well as for the maintenance of productive ability and vision in the dark. Vitamin A especially plays a key role on the maintenance of the epithelia integrity and activation. Therefore, it can promote the epithelia activation and keep skin bright and elastic. So Vitamin A was generally used as the biologically active ingredient in cosmetics from domestic or international cosmetic companies.

But there are some defects of Vitamin A as a kind of dermal medicine such as instability due to many unsaturated double-bonds in its chemical structure, low permeability due to its great molecular weight, and the liposolubility by which Vitamin A should be packed in the hydrophilic carrier for usage.

A characteristic of Liposome is its micro-vesicle structure which is composed of double lipid molecules. The microstructure can improve the stability of the sealed medicine, promote the endermic absorption, prolong the effect time, reduce side effects of the medicine, and has the ability to guide the medicine to the pathological areas. Therefore, Liposome was applied extensively to pharmaceutical and cosmetics production. Vitamin A Liposomes can improve the stability of Vitamin A, promote the ability to permeate skin and the solubility in water. Now, Vitamin A Liposome, as well as the related cosmetics has already become focus of study.

It was by far reported that the Vitamin A Liposomes are all the common Liposomes, namely, Liposomes Suspension. There are actually many defects of the common Vitamin A Liposomes as follows.

1. The Liposomes Suspension as colloid solution lacks of thermodynamic stability. So it is easy for the Liposomes Suspension to conglomerate, amalgamate and sedimentate in the aqueous solution. In addition, due to the oxidative cleavage, the leakage of the sealed medicine also causes the instability of the common Liposomes.

2. The marked instability of the medicine containing Vitamin A in the aqueous solution is due to the structure instability of the Vitamin A.

3. The Vitamin A Liposome Suspension has commonly the fixed Vitamin A content. Furthermore, the different Vitamin A content is required for different cosmetics production. So it is inconvenient to produce cosmetics containing Vitamin A by using the Vitamin A Liposomes Suspension because of the definite proportion of Liposome in the cosmetics with Vitamin A.

Therefore, it is very important to seek for a new method by which Vitamin A Liposomes and the related medicine become stable for long-term preservation and conveniently producing cosmetics with Vitamin A.

An object of the present invention is to provide a new kind of Vitamin A Liposomes, which not only improves the stability of Vitamin A and Liposome but also is more convenient for cosmetics production.

Another object of the present invention is to provide a new method of Vitamin A Liposome preparation.

### SUMMARY OF THE INVENTION

The sealed Vitamin A serves essentially as biologically active ingredient in the new vitamin A Liposomes provided by this invention and which contains support substance and the lipid ingredients serving as the accessories and the membranes.

The method of vitamin A Liposomes preparation is as follows. The solid Vitamin A pro-Liposome is made from Vitamin A and the lipid ingredients by adding the support substance. According to your needs, Vitamin A Liposomes can be obtained through hydration and vibration by adding water into the Vitamin A pro-Liposomes before usage.

### DETAILED DESCRIPTION OF THE INVENTION

This invention openly provides a new kind of Vitamin A Liposomes, which not only improve the stability of Vitamin A and Liposome but also is more convenient for production of the cosmetics with the sound formula.

The vitamin A Liposomes produced through this invention contains vitamin A serving as its active ingredient, and the support substance and the lipid ingredients as the accessories and the membranes. In the Vitamin A Liposomes the content of vitamin A is 0.1-20%, and the support substance 2-40%. The remainders are the lipid ingredients, buffer and water.

In the vitamin A Liposomes produced through this invention the support substance is selected from one or several sorts of materials as follows: Mannitol, Sorbitol, Glucose, Sucrose, Lactose, Mycose, Sodium chloride, polyvinyl pyrrolidone, etc.

In the vitamin A Liposomes produced through this invention the lipid ingredient is selected from one or several sorts of material as follows: Soya lecithin, Yolk lecithin, Distearoyl phosphatidyl choline, Dipalmitoyl Phosphatidyl Choline, Poloxamer, Dimyristoyl Phosphatidyl-choline, Ceramide, Nonionic Surfactant Brij, Cholesterol, etc.

This invention published the method of vitamin A Liposomes preparation. The solid Vitamin A pro-Liposome is made from Vitamin A and lipid ingredients by adding the support substance. Then, Vitamin A Liposomes can be produced by adding water into the Vitamin A pro-Liposomes. The Vitamin A pro-Liposomes is a kind of the granular and dry solid agent which can be converted into the Vitamin A Liposomes through hydration and vibration by adding water into the Vitamin A pro-Liposomes before usage.

The method of the Vitamin A pro-Liposomes preparation in this invention is as follows:

(1) The lipid solution can be obtained when Vitamin A and the lipid ingredients are melted by heating or dissolved by the organic solvent.

(2) The above-mentioned lipid solution is sprayed upon the support substance suspending in the fluidized bed. The dry Vitamin A pro-Liposomes can be obtained after volatilization of the organic solvent. In addition, the Vitamin A Liposomes with the support substance can be also obtained from the lipid solution with Vitamin A and the aqueous solution with the support substance through the method of the film dispersion or Fusion or Filling. The Vitamin A pro-Liposomes can be obtained after the Vitamin A Liposomes is dehydrated by freeze-drying or Spray-drying.

In the Vitamin A pro-Liposomes the content of vitamin A is 0.2-40%. The content of vitamin A is 0.1-20% in the Vitamin A Liposome which is obtained by adding water into the Vitamin A pro-Liposomes.

The proportion of the support substance is 1-80% in the Vitamin A pro-Liposomes, and 2-40% in the Vitamin A Liposomes.

Apart from these advantages of the common liposomes, such as improving the stability of Vitamin A, enhancing the endermic absorption, prolonging the effect time of drugs, the Vitamin A liposome produced through the method of the Vitamin A pro-liposomes preparation in this invention possesses advantages as follows:

1. Improving the stability of Vitamin A liposomes. Because the pro-liposome is solid, it has no defects of instability of the common liposomes, such as conglomeration, sedimentation, amalgamation and leakage, etc. The Vitamin A pro-liposomes can be preserved for a long term. Vitamin A liposomes can be obtained through hydration and vibration by adding water into the Vitamin A pro-Liposomes before usage.

2. Improving the stability of Vitamin A. The Vitamin A pro-liposomes produced through this method is solid. The stability of the solid Vitamin A serving as the active ingredient is greater than the liquid Vitamin A.

3. Being mixed with other ingredients in the random proportion. It is simple and convenient to produce the cosmetics containing Vitamin A by using the Vitamin A liposomes produced through this method as materiel. There is the definite range of Liposomes volume percentage in the cosmetics with liposomes. The property of cosmetics, such as viscosity, fluidity, consistance, the active ingredient content, etc, would be influenced out of the volume percentage range. It is inconvenient to produce cosmetics containing Vitamin A by using the common Liposomes because of the definite volume percentage of Liposomes in the cosmetics with liposomes. So the different Vitamin A content is required for production of the different cosmetics.

The liposomes with the different Vitamin A content can be obtained by using the above-mentioned Vitamin A pro-liposomes through regulation of the added water volume before usage. The liposomes with the different Vitamin A content produced through this method can meet the different demands of the cosmetics formulas.

The experiment about the stability showed that the stability of the Vitamin A pro-liposomes is more dependable as compared with the common liposomes. Three groups of the Vitamin A pro-liposomes and the common Vitamin A liposomes were preserved in condition of 40 □ and 75% relative humidity respectively. The Vitamin A contents of all samples were measured with high performance liquid chromatography respectively at 0, 1, 2 and 3 months. The contents of Vitamin A in the Vitamin A pro-liposomes and the common Vitamin A liposomes at the 0 month served as 100%. The content percentages were obtained when the contents at the other months were compared with the contents at the 0 month. The results showed that the Vitamin A contents in the common Vitamin A liposomes gradually decreased with prolongation of the preservation time, but the Vitamin A contents in the Vitamin A pro-liposomes only have a little change. Therefore, the Vitamin A pro-liposome has the ability to improve the stability of Vitamin A serving as the active ingredient.

TABLE 1
Comparison of the stability of Vitamin A between in the
pro-liposomes and in the common liposomes (n = 3)
The content of Vitamin A (%)

| | | | | |
|---|---|---|---|---|
| Time (Mon) | 0 | 1 | 2 | 3 |
| Common liposomes | 100.00 | 90.24 | 87.12 | 76.33 |
| Pro-liposomes | 100.00 | 99.98 | 100.05 | 97.80 |

The Vitamin A pro-liposomes produced through this method can be used in the production of drugs and cosmetics containing Vitamin A.

### THE PREFERRED EMBODIMENT

Our invention was illustrated with 3 examples as follows. These illustrations do not mean any restriction to this invention.

### Example 1

Materials: Vitamin A 10 g, Lecithin of soybeans 30 g, Cholesterol 30 g, Poloxamer F₆₈ 40 g, Glucose 200 g, Chloroform 200 ml, Phosphoric acid buffer (pH 7.4) 1000 ml.

Vitamin A, soy lecithin, poloxamer F₆₈ and cholesterol were put into a round bottom flask (10 liter) and dissolved with chloroform, and then the flask was put into the constant temperature water bath (25-40° C.) for the Rotated Thin-Film Evaporation. A lipid membrane was obtained on the bottom of the flask after evaporation and reserved for using. Glucose was dissolved with 800 ml Phosphoric acid buffer (pH 7.4), and then the solution was filtered. The filtrate was poured into the flask with the lipid membrane. After hydration and vibration of the mixed solution, Phosphoric acid buffer (pH 7.4) was added into the mixed solution to 1000 ml. Liposomes Suspension was obtained through the ultrasonic processing (output 4, duty cycle 50%, time 10 mins). After freeze-drying (temperature −50 □, vacuity 20-100 millitorr), the loose Vitamin A pro-liposome was obtained. Vitamin A Liposomes can be obtained through vibration by adding the distilled water into the Vitamin A pro-Liposomes, according to your needs, before usage.

### Example 2

Materials: Vitamin A 100 g, Yolk lecithin 50 g, Cholesterol 50 g, Sucrose 40 g, Phosphoric acid buffer (pH 7.4) 1000 ml.

Vitamin A, yolk lecithin, cholesterol were put into a Conical Erlenmeyer Flask and were melted by heating or dissolved with the organic solvent The flask with the lipid solution was put into the constant temperature water bath (80° C.) for using. Sucrose (40 g) was dissolved with 800 ml Phosphoric acid buffer (pH 7.4), and then the solution was filtered. The filtrate was heated to the same temperature as the lipid solution and mixed with the lipid solution through vibration. Phosphoric acid buffer (pH 7.4) was added into the mixed solution to 1000 ml after refrigeration of the mixed solution. Liposomes Suspension was obtained through the high pressure homogenizing (the range of pressure, 50 MPa-10 MPa). After the Spray-drying, the Vitamin A pro-liposomes with better liquidity was obtained. Vitamin A Liposomes can be obtained through vibration by adding the distilled water into the Vitamin A pro-Liposomes, according to your needs, before usage.

### Example 3

Materials: Vitamin A 50 g, polydioxyethylene hexadecyl ether [symbol] [symbol] 60 g, Cholesterol 40 g, Poloxamer F₆₈ 50 g, Mycose 80 g, Ethyl ether 200 ml, Phosphoric acid buffer (pH 7.4) 1000 ml.

Vitamin A, polydioxyethylene hexadecyl ether [symbol] [symbol] poloxamer F68, cholesterol were put into a Conical Erlenmeyer flask (500 ml) and dissolved with Ethyl ether for using. Mycose (80 g) was dissolved with 800 ml Phosphoric acid buffer (pH 7.4), and then the solution was filtered. The filtrate was poured into the flask with the lipid solution. After volatilization of organic solvent, Liposomes Suspension was obtained through the magic stirring (stirring speed, 200-1000 rpm) in the constant temperature water bath (30˜60 □). After freeze-drying (temperature −50 □, vacuity 20-100 millitorr), the loose Vitamin A pro-liposome was obtained. Vitamin A Liposomes can be obtained through vibration by adding the distilled water into the Vitamin A pro-Liposomes, according to your needs, before usage.

The above-mentioned examples are merely used to illustrate the preferred embodiment in our invention. Technicians in this field are allowed to modify and change these methods, which does not depart from the spirit and range of this invention. The attached claims cover all of those modifications in the range of this invention.

1-6. (canceled)

7. A Vitamin A liposome, comprising:
Vitamin A serving as an active ingredient, and the support substance and the lipid ingredients serving as the accessories and the membranes; characterized in that:
the content of Vitamin A is 0.1-20%, and the support substance 2-40%, the remainders are the lipid ingredients, buffer and water.

8. The Vitamin A Liposome according to claim 7, wherein the support substance is selected from one or several sorts of materials as follows: Mannitol, Sodium chloride, polyvinyl pyrrolidone, etc.

9. The Vitamin A Liposome according to claim 7, wherein the lipid ingredient is selected from one or several sorts of materials as follows: Yolk lecithin, Distearoylphosphatidyl choline, Dipalmitoyl Phosphatidyl Choline, Poloxamer, Dimyristoyl Phosphatidyl-choline, Nonionic Surfactant Brij, etc.

10. A method of Vitamin A Liposomes preparation, characterized in that: the solid Vitamin A pro-liposome is made from Vitamin A and the lipid ingredients by adding the support substance; according to your needs, the Vitamin A Liposomes can be obtained through hydration and vibration by adding water into the Vitamin A pro-Liposomes before usage.

11. The method of Vitamin A Liposomes preparation according to claim 10, wherein the content of Vitamin A in the Vitamin A pro-Liposomes is 0.2-40%, and the support substance 1-80%, the remainders are the lipid ingredients.

12. The method of Vitamin A Liposomes preparation according to claim 11, wherein the process of Vitamin A pro-Liposomes preparation is as follows:
(1) The lipid solution can be obtained when Vitamin A and the lipid ingredients are melted by heating or dissolved by the organic solvent; and
(2) The above-mentioned lipid solution is sprayed upon the support substance suspending in the fluidized bed, the dry Vitamin A pro-Liposomes can be obtained after volatilization of the organic solvent; in addition, the Vitamin A Liposomes with the support substance can be also obtained from the lipid solution with Vitamin A and the aqueous solution with the support substance through the method of the film dispersion or Fusion or Filling, the Vitamin A pro-Liposomes can be obtained after the Vitamin A Liposomes is dehydrated by freeze-drying or Spray-drying.
